(19) United States
(12) Patent Application Publication
(10) Pub. No.: US 2008/0317685 A1
(43) Pub. Date: Dec. 25, 2008

(54) Photoprotective cosmetic compositions comprising photostabilized dibenzoylmethane compounds and siloxane-containing arylalkyl benzoate amide compounds

(75) Inventors: Didier Candau, Bievres (FR); Benoit Muller, Paris (FR); Herve Richard, Les Pavillons Sous Bois (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/987,110

(22) Filed: Nov. 27, 2007

Related U.S. Application Data
(60) Provisional application No. 60/873,565, filed on Dec. 8, 2006.

(30) Foreign Application Priority Data
Nov. 27, 2006 (FR) 06/55121

(51) Int. Cl. A61K 8/42; A61Q 17/04; A61Q 5/00; A61Q 3/02

(52) U.S. Cl. 424/59; 424/61; 424/70.9

(57) ABSTRACT

Photostable, topically applicable cosmetic/dermatological compositions contain at least one dibenzoylmethane sunscreen compound and at least one photostabilizing siloxane-containing arylalkyl amide compound of formula (I) below:

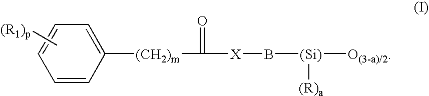

### CROSS-REFERENCE TO PRIORITY/PROVISIONAL APPLICATIONS

This application claims priority under 35 U.S.C. § 119 of FR 06/55121, filed Nov. 27, 2006, and of U.S. Provisional Application No. 60/873,565, filed Dec. 8, 2006, each hereby expressly incorporated by reference and each assigned to the assignee hereof.

### BACKGROUND OF THE INVENTION

1. Technical Field of the Invention

The present invention relates to cosmetic compositions containing the combination of at least one dibenzoylmethane derivative and at least one particular siloxane-containing arylalkyl benzoate amide derivative of formula (I) as defined below.

This invention also relates to a process for the photostabilization, with respect to radiation, of at least one dibenzoylmethane derivative by an effective amount of at least one particular siloxane-containing arylalkyl benzoate amide derivative of formula (I), as defined below.

The present invention also relates to the formulation of said particular siloxane-containing arylalkyl benzoate amide derivatives of formula (I) into compositions comprising, in a cosmetically acceptable support, at least one screening agent of the dibenzoylmethane derivative type for the purpose of improving the effectiveness of said composition with respect to UV-A rays.

2. Description of Background and/or Related and/or Prior Art

Light radiation with wavelengths in the range 280 nm to 400 nm is known to brown the human epidermis; more particularly, rays with a wavelength in the range 280 to 320 nm, known as UV-B rays, are known to cause erythema and skin burns that may be deleterious to the development of a natural tan. For these reasons and for aesthetic reasons, there exists a constant demand for means for controlling this natural tanning which can thereby control the color of the skin; it is therefore advisable to screen out said UV-B radiation.

It is also known that UV-A rays with wavelengths in the range 320 to 400 nm, which cause the skin to brown, are capable of inducing alterations in said skin, especially in the case of sensitive skin or of skin continually exposed to solar radiation. UV-A rays in particular cause a loss of elasticity of the skin and the appearance of wrinkles, resulting in premature aging of the skin. These promote triggering of the erythematosus reaction or amplify this reaction in certain individuals and may even be the cause of phototoxic or photo-allergic reactions. Thus, for aesthetic and cosmetic reasons, such as preserving the natural elasticity of the skin, for example, more and more individuals would prefer to control the effect of UV-A rays on their skin. Thus, it is also desirable to screen out UV-A radiation.

With the goal of providing protection for the skin and keratinous substrates against UV radiation, sunscreen compositions comprising organic screening agents, which are active in the UV-A range and active in the UV-B range, are generally employed. The majority of these screening agents are liposoluble.

In this regard, a particularly advantageous family of UV-A screening agents currently consists of dibenzoylmethane derivatives, and especially 4-tert-butyl-4′-methoxydibenzoylmethane, which in fact have an extremely good intrinsic absorbing capacity. These dibenzoylmethane derivatives, which are now products that are well-known per se as screening agents that are active in the UV-A range, are in particular described in FR-A-2,326,405 and FR-A-2,440,933, and also in EP-A-0,114,607; 4-tert-butyl-4′-methoxydibenzoylmethane is furthermore currently marketed under the trademark PARSOL 1789® by DSM Nutritional Products.

Unfortunately, it has been discovered that dibenzoylmethane derivatives are products that are relatively sensitive to ultraviolet (especially UV-A) radiation, that is to say more specifically that they have an annoying tendency to degrade more or less rapidly under the action of said radiation. Thus, this substantial lack of photochemical stability of the dibenzoylmethane derivatives in the face of the ultraviolet radiation to which they are, by nature, intended to be subjected cannot guarantee constant protection during prolonged exposure to the sun, so that repeated applications, at regular, close intervals have to be made by the user in order to effectively protect the skin against UV rays.

### SUMMARY OF THE INVENTION

It has now surprisingly been discovered that by combining with the dibenzoylmethane compounds mentioned above, an effective amount of a particular arylalkyl amide derivative of formula (I) that will be defined further below, it is possible to substantially and notably further improve the photochemical stability (or photostability) of these same dibenzoylmethane derivatives and their effectiveness in the UV-A range. The compositions containing such a combination also provide, after application, a more homogeneous distribution of the UV-screening agent.

These discoveries constitute the basis of the present invention.

Thus, the present invention features compositions comprising, formulated into a cosmetically acceptable support, at least one UV-screening system which comprises:

(a) at least one UV-A-screening agent of the dibenzoylmethane derivative type; and

(b) at least one siloxane-containing arylalkyl benzoate amide derivative of formula (I), as defined below.

The present invention also features a process for improving the chemical stability with respect to UV radiation of at least one UV-A screening agent of the dibenzoylmethane derivative type, by combining with said dibenzoylmethane derivative, an effective amount of at least one siloxane-containing arylalkyl benzoate amide derivative of formula (I), as defined below.

Finally, the present invention also features the use of at least one siloxane-containing arylalkyl benzoate amide derivative of formula (I) in a composition comprising, formulated into a cosmetically acceptable support, an effective amount of at least one dibenzoylmethane derivative for the purpose of improving the effectiveness of said composition with respect to UV-A rays.

Other features, aspects and advantages of the invention will become apparent from the detailed description which follows.

The term “cosmetically acceptable” means compatible with the skin and/or its integuments, which has a pleasant color, odor and feel and which does not cause any unacceptable discomfort (stinging, tautness, red blotches), liable to discourage consumers from using this composition.

The term “effective amount” means an amount sufficient to obtain a notable and significant improvement in the photostability of the dibenzoylmethane derivative or derivatives in the cosmetic composition. This minimum amount of compound of formula (I), which may vary depending on the nature of the support retained for the composition may be determined without any difficulty by means of a conventional photostability measurement test such as that provided in the examples below.

The siloxane-containing arylalkyl amide derivatives according to the invention are selected from among those having the following general formula (I):

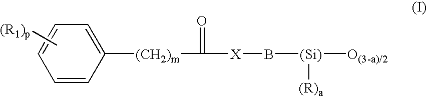

in which:

R₁ is a linear or branched C₁-C₄ alkyl radical;

p is 0-2;

m is 0-4;

X is —N(R₂)—;

R₂ is H, a linear or branched C₁-C₂₀ alkyl radical, the acetyl group or the group —CH₂CH₂NH(C═O)Ph;

R, being identical or different, are each a linear or branched C₁-C₂₀ alkyl radical, a C₆-C₁₂ aryl radical, 3,3,3-trifluoropropyl, trimethylsilyloxy or a linear or branched C₁-C₁₀ alkoxy radical;

a=1-2;

B is a divalent radical selected from among those having one of the formulae (II), (III) or (IV) below:

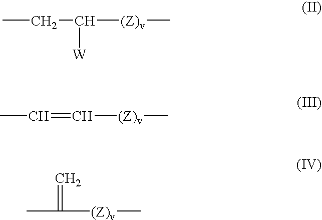

in which:

Z is a linear or branched, saturated or unsaturated C₁-C₆ alkylene radical, optionally substituted by a hydroxyl radical or a linear or branched, saturated or unsaturated C₁-C₈ alkyl radical;

W is a hydrogen atom, a hydroxyl radical or a linear or branched, saturated or unsaturated C₁-C₈ alkyl radical; and

v is 0 or 1.

In addition to the units of formula —B—(Si)(R)ₐ(O)_{(3-a)/2}, the organosiloxane may comprise units of formula (R)_{b}—(Si)(O)_{(4-b)/2} in which:

R has the same definition given in the formula (I) above; and

b=2-3.

### DETAILED DESCRIPTION OF BEST MODE AND SPECIFIC/PREFERRED EMBODIMENTS OF THE INVENTION

Preferably, the groups —(Si)(R)ₐ(O)_{(3-a)/2} have the formulae (V) or (VI) below:

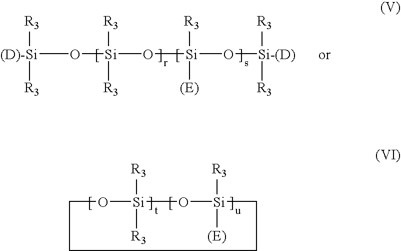

in which:

(E) is a bond from the silicone-based chain and the group B in the formula (I) is as defined above;

R₃ being identical or different, are selected from among the linear or branched C₁-C₂₀ alkyl radicals, phenyl, 3,3,3-trifluoropropyl and trimethylsilyloxy radicals, at least 80% by number of the R₃ radicals being methyl;

(D) being identical or different, are selected from among the R₃ radicals and the (E) radical;

r is an integer from 0 and 200 inclusively, and s is an integer from 0 to 50 inclusively, and when s=0, at least one of the two symbols (D) is B,

u is an integer from 1 to 10 inclusively, and t is an integer from 0 to 10 inclusively, it being understood that t+u is greater than or equal to 3, with the proviso that when q is 1, then u is equal to 1, s is equal to 1 and the symbol (D) cannot denote B or s is equal to 0 and only one of the symbols (D) is B.

In the formulae (I) to (VI), among the alkyl groups, exemplary are the methyl, ethyl, n-propyl, isopropyl, n-butyl, isobutyl, tert-butyl, 2-ethylhexyl, dodecyl or hexadecyl radicals.

The linear or cyclic diorganosiloxanes of formula (V) or (VI) according to the present invention are random oligomers or polymers preferably having at least one, and more preferably all of the following characteristics:

R₃ is preferably methyl, and

(D) is preferably methyl (in the case of linear compounds of formula (V)).

As examples of particularly preferred compounds of formula (I), representative are the following compounds of formulae (a) to (e):

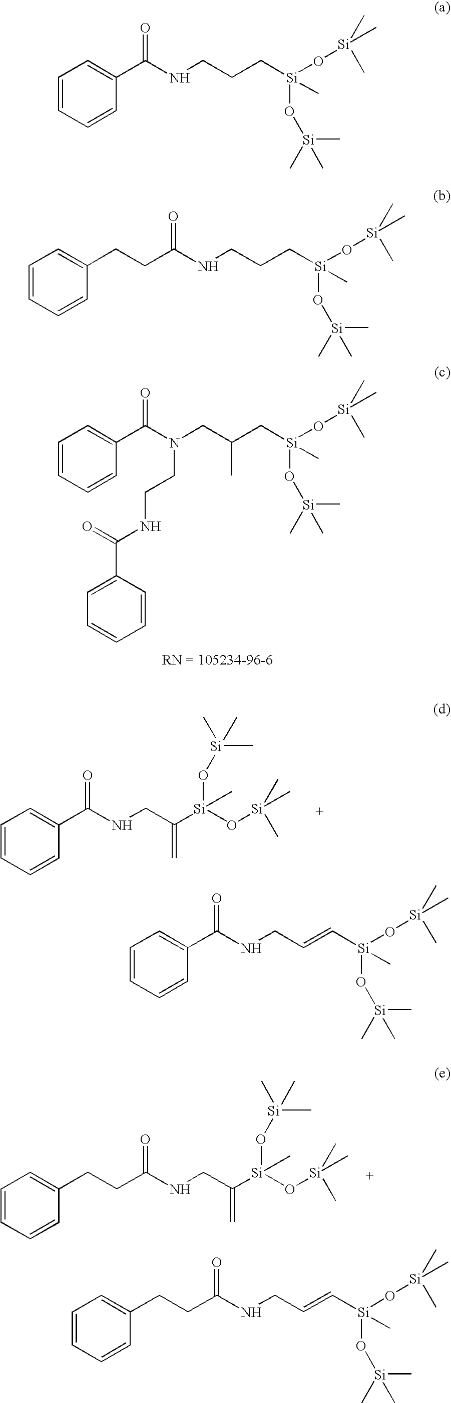

The alkyl amide acyl ester derivative (compound C) is described in U.S. Pat. No. 6,528,068.

The amide derivatives of formulae (I) with X═—NH— may be prepared by starting from the acid derivative of formula (VII) with in situ formation of the corresponding acid chloride which is reacted with the siloxane-containing amine derivative of formula (VIII) according to the following scheme:

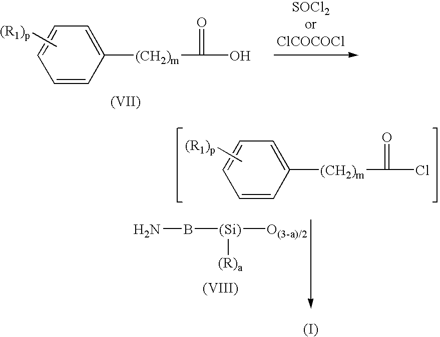

with the R, R₁, p, m, a and B radicals having the same definitions as above.

The SiH groups may be present in the chain and/or at the ends of the chain. These SiH-containing derivatives are well-known products in the silicone industry and are generally commercially available. They are, for example, described in U.S. Pat. Nos. 3,220,972, 3,697,473 and 4,340,709.

The derivatives of formula (I) according to the invention are preferably each present in the compositions in amounts of 1 to 30% by weight and more preferably of 3 to 20% by weight relative to the total weight of the composition.

Among the dibenzoylmethane derivatives, particularly exemplary are:
2-methyldibenzoylmethane;
4-methyldibenzoylmethane;
4-isopropyldibenzoylmethane;
4-tert-butyldibenzoylmethane;
2,4-dimethyldibenzoylmethane;
2,5-dimethyldibenzoylmethane;
4,4′-diisopropyldibenzoylmethane;
4,4′-dimethoxydibenzoylmethane;
4-tert-butyl-4′-methoxydibenzoylmethane;
2-methyl-5-isopropyl-4′-methoxydibenzoylmethane;
2-methyl-5-tert-butyl-4′-methoxydibenzoylmethane;
2,4-dimethyl-4′-methoxydibenzoylmethane; and
2,6-dimethyl-4-tert-butyl-4′-methoxydibenzoylmethane.

Among the dibenzoylmethane derivatives mentioned above, use will in particular be made of 4-isopropyldibenzoylmethane, marketed under the trademark EUSOLEX 8020 by Merck, and corresponding to the following formula:

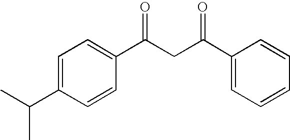

Most particularly, it is preferred to use 4-(tert-butyl)-4′-methoxydibenzoylmethane or Butyl Methoxy Dibenzoylmethane marketed under the trademark PARSOL 1789 by DSM Nutritional Products; this screening agent corresponds to the following formula:

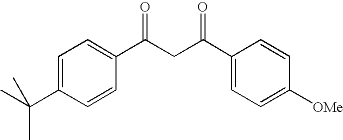

The dibenzoylmethane derivative or derivatives may be present in the compositions according to the invention in amounts which preferably vary from 0.01 to 10% by weight and more preferably from 0.1 to 6% by weight, relative to the total weight of the composition.

The compositions according to the invention may, in addition, comprise other additional organic or inorganic UV-screening agents that are active in the UV-A range and/or the UV-B range, that are water-soluble or liposoluble or else insoluble in the cosmetic solvents commonly employed.

Of course, one skilled in the art will take care to select the optional additional screening agent or agents and/or their amounts so that the advantageous properties intrinsically linked to the compositions according to the invention are not, or are not substantially, impaired by the envisaged addition or additions, especially the improvement of the photostability of the dibenzoylmethane derivative.

The additional organic photoprotective agents are especially selected from anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives, especially those cited in U.S. Pat. No. 5,624,663; benzimidazole derivatives; imidazolines; bis(benzoazolyl) derivatives, as described in EP 669323 and U.S. Pat. No. 2,463,264; p-aminobenzoic acid (PABA) derivatives; methylenebis(hydroxyphenylbenzotriazole) derivatives, as described in U.S. Pat. Nos. 5,237,071, 5,166,355, GB-2303549, DE-197, 26, 184 and EP-893, 119; benzoxazole derivatives as described in EP-0,832,642, EP-1,027,883, EP-1,300,137 and DE-10162844; screening polymers and screening silicones, such as those described, in particular in WO-93/04665; α-alkylstyrene-derived dimers, such as those described in DE-19855649; 4,4-diarylbutadienes such as described in EP-0,967,200, DE-19746654, DE-9755649, EP-A-1,008,586, EP-1,133,980 and EP-133,981 and mixtures thereof.

As examples of additional organic photoprotective agents, representative are those denoted below under their INCI name:

Para-Aminobenzoic Acid Derivatives:

### PABA;

### Ethyl PABA;

### Ethyl Dihydroxypropyl PABA;

Ethylhexyl Dimethyl PABA marketed in particular under the trademark

### ESCALOL 507 by ISP;

### Glyceryl PABA; and

PEG-25 PABA marketed under the trademark UVINUL P25 by BASF.

Salicylic Derivatives:

Homosalate marketed under the trademark Eusolex HMS by Rona/EM Industries;
Ethylhexyl Salicylate marketed under the trademark NEO HELIOPAN OS by Haarmann and Reimer;
Dipropyleneglycol Salicylate marketed under the trademark DIPSAL by Scher; and
TEA Salicylate marketed under the trademark NEO HELIOPAN TS by Haarmann and Reimer.

Cinnamic Derivatives:

Ethylhexyl Methoxycinnamate marketed, in particular under the trademark PARSOL MCX by Hoffmann La Roche;

### Isopropyl Methoxycinnamate;

Isoamyl Methoxycinnamate marketed under the trademark NEO HELIOPAN E 1000 by Haarmann and Reimer;

### Cinoxate;

### DEA Methoxycinnamate;

### Diisopropyl Methylcinnamate; and

### Glyceryl Ethylhexanoate Dimethoxycinnamate.

β,β-Diphenyl Acrylate Derivatives:

Octocrylene marketed, in particular, under the trademark UVINUL N539 by BASF; and
Etocrylene, marketed, in particular under the trademark UVINUL N35 by BASF.

Benzophenone Derivatives:

Benzophenone-1 marketed under the trademark UVINUL 400 by BASF;
Benzophenone-2 marketed under the trademark UVINUL D50 by BASF;
Benzophenone-3 or Oxybenzone marketed under the trademark UVINUL M40 by BASF;
Benzophenone-4 marketed under the trademark UVINUL MS40 by BASF;

### Benzophenone-5;

Benzophenone-6 marketed under the trademark Helisorb 11 by Norquay;
Benzophenone-8 marketed under the trademark Spectra-Sorb UV-24 by American Cyanamid;
Benzophenone-9 marketed under the trademark UVINUL DS-49 by BASF;

### Benzophenone-12; and

n-Hexyl 2-(4-diethylamino-2-hydroxybenzoyl)benzoate marketed under the trademark “UVINUL A+” or in form of mixture with Ethylhexyl Methoxycinnamate as the product marketed under the trademark <<UVINUL A+B>> by BASF.

Benzylidene Camphor Derivatives:

3-Benzylidene camphor manufactured under the trademark MEXORYL SD by Chimex;
4-Methylbenzylidene camphor marketed under the trademark EUSOLEX 6300 by Merck;
Benzylidene Camphor Sulfonic Acid manufactured under the trademark MEXORYL SL by Chimex;
Camphor Benzalkonium Methosulfate manufactured under the trademark MEXORYL SO by Chimex;
Terephthalylidene Dicamphor Sulfonic Acid manufactured under the trademark MEXORYL SX by Chimex; and
Polyacrylamidomethyl Benzylidene Camphor manufactured under the trademark MEXORYL SW by Chimex.

Phenylbenzimidazole Derivatives:

Phenylbenzimidazole Sulfonic Acid marketed, in particular under the trademark EUSOLEX 232 by Merck; and
Disodium Phenyldibenzimidazole Tetrasulfonate marketed under the trademark NEO HELIOPAN AP by Haarmann and Reimer.

Phenylbenzotriazole Derivatives:

Drometrizole Trisiloxane marketed under the trademark SILATRIZOLE by Rhodia Chimie; and
Methylene bis-Benzotriazolyl Tetramethylbutylphenol marketed in solid form under the trademark MIXXIM BB/100 by Fairmount Chemical or in micronized form in aqueous dispersion under the trademark TINOSORB M by Ciba Speciality Chemicals.

Triazine Derivatives:

Bis-Ethylhexyloxyphenol Methoxyphenyl Triazine marketed under the trademark TINOSORB S by Ciba-Geigy;
Ethylhexyl triazone marketed, in particular under the trademark UVINUL T150 by BASF;
Diethylhexyl Butamido Triazone marketed under the trademark UVASORB HEB by Sigma 3V;
2,4,6-tris(dineopentyl 4′-aminobenzalmalonate)-s-triazine;
2,4,6-tris(diisobutyl 4′-aminobenzalmalonate)-s-triazine; and symmetrical triazine screening agents described in U.S. Pat. No. 6,225,467, WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which are described in WO 06/035000, WO 06/034982, WO 06/034991, WO 06/035007, WO 2006/034992 and WO 2006/034985.

Anthranilic Derivatives:

Menthyl anthranilate marketed under the trademark NEO HELIOPAN MA by Haarmann and Reimer.

Imidazoline Derivatives:

### Ethylhexyl Dimethoxybenzylidene Dioxoimidazoline Propionate.

Benzalmalonate Derivatives:

Dineopentyl 4′-methoxybenzalmalonate; and
Polyorganosiloxane having benzalmalonate functional groups such as Polysilicone-15 marketed under the trademark PARSOL SLX by Hoffmann La Roche.

4-4-Diarylbutadiene Derivatives:
1,1-dicarboxy-(2,2′-dimethylpropyl)-4,4-diphenylbutadiene.

Benzoxazole Derivatives:

2,4-bis[5-1 (dimethylpropyl)benzoxazol-2-yl-(4-phenyl)-imino]-6-(2-ethylhexyl)-imino-1,3,5-triazine marketed under the trademark UVASORB K2A by Sigma 3V; and
mixtures thereof.

The preferred additional organic photoprotective agents are selected from among:
Ethylhexyl Methoxycinnamate;
Homosalate;
Ethylhexyl Salicylate;
Octocrylene;
Phenylbenzimidazole Sulfonic Acid;
Benzophenone-3;
Benzophenone-4;
Benzophenone-5;
n-Hexyl 2-(4-diethylamino-2-hydroxybenzoyl)benzoate;
4-Methylbenzylidene camphor;
Terephthalylidene Dicamphor Sulfonic Acid;
Disodium Phenyl Dibenzimidazole Tetrasulfonate;
Methylene bis-Benzotriazolyl Tetramethylbutylphenol;
Ethylhexyl triazone,
Bis-Ethylhexyloxyphenol Methoxyphenyl Triazine;
Diethylhexyl Butamido Triazone;
2,4,6-Tris(dineopentyl 4′-aminobenzalmalonate)-s-triazine;
2,4,6-Tris(diisobutyl 4′-aminobenzalmalonate)-s-triazine;
2,4,6-Tris(biphenyl-4-yl-1,3,5-triazine);
2,4,6-Tris(terphenyl)-1,3,5-triazine;
Drometrizole Trisiloxane;
Polysilicone-15;
Dineopentyl 4′-methoxybenzalmalonate
1,1-Dicarboxy-(2,2′-dimethylpropyl)-4,4-diphenylbutadiene;
2,4-Bis[5-1 (dimethylpropyl)benzoxazol-2-yl-(4-phenyl)-imino]-6-(2-ethylhexyl)-imino-1,3,5-triazine;
and mixtures thereof.

The inorganic photoprotective agents are selected from among coated or uncoated metal oxide pigments (average size of the primary particles: generally from 5 nm to 100 nm, preferably from 10 nm to 50 nm) such as, for example pigments of titanium oxide (amorphous or crystallized in rutile and/or anatase form), iron oxide, zinc oxide, zirconium oxide or cerium oxide, which are all UV photoprotective agents well-known per se.

The pigments may be coated or uncoated.

The coated pigments are pigments which have undergone one or more surface treatments of chemical, electronic, mechanochemical and/or mechanical nature with compounds as described, for example, in Cosmetics & Toiletries, February 1990, Vol. 105, p. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen, elastin), alkanolamines, silicon oxides, metal oxides or sodium hexametaphosphate.

In a known manner, silicones are organosilicon polymers or oligomers having a linear or cyclic structure, that are branched or crosslinked, of variable molecular weight, obtained by polymerization and/or polycondensation of appropriately functionalized silanes, and composed for the main part by a repetition of main units in which the silicon atoms are joined together by oxygen atoms (siloxane bond), optionally substituted hydrocarbon-based radicals being directly bonded via a carbon atom to said silicon atoms.

The term “silicones” also encompasses the silanes required for their preparation, in particular, alkylsilanes.

The silicones used for coating pigments suitable for the present invention are preferably selected from among alkylsilanes, polydialkylsiloxanes and polyalkylhydrogensiloxanes. Even more preferably, the silicones are selected from among octyltrimethylsilane, polydimethylsiloxanes and polymethylhydrogensiloxanes.

Of course, the metal oxide pigments, prior to their treatment with silicones, may have been treated by other surface agents, in particular by cerium oxide, alumina, silica, aluminum compounds, silicon compounds, or mixtures thereof.

The coated pigments are more particularly titanium oxides coated with:

silica, such as the product SUNVEIL from Ikeda and the product EUSOLEX T-AVO from Merck;

silica and iron oxide such as the product SUNVEIL F from Ikeda;

silica and alumina such as the products MICROTITANIUM DIOXIDE MT 500 SA and MICROTITANIUM DIOXIDE MT 100 SA from Tayca, TIOVEIL from Tioxide and MIRASUN TiW 60 from Rhodia;

alumina such as the products TIPAQUE TTO-55(B) and TIPAQUE TTO-55(A) from Ishihara and UVT 14/4 from Kemira;

alumina and aluminum stearate such as the product MICROTITANIUM DIOXIDE MT 100 TV, MT 100 TX, MT 100 Z, MT-01 from Tayca, the products SOLAVEIL CT-10 W, SOLAVEIL CT 100 and SOLAVEIL CT 200 from Uniqema;

silica, alumina and alginic acid such as the product MT-100 AQ from Tayca;

alumina and aluminum laurate such as the product MICROTITANIUM DIOXIDE MT 100 S from Tayca;

iron oxide and iron stearate such as the product MICROTITANIUM DIOXIDE MT 100 F from Tayca;

zinc oxide and zinc stearate such as the product BR351 from Tayca;

silica and alumina and treated with a silicone such as the products MICROTITANIUM DIOXIDE MT 600 SAS, MICROTITANIUM DIOXIDE MT 500 SAS or MICROTITANIUM DIOXIDE MT 100 SAS from Tayca;

silica, alumina, aluminum stearate and treated with a silicone such as the product STT-30-DS from Titan Kogyo;

silica and treated with a silicone such as the product UV-TITAN X 195 from Kemira or the product SMT-100 WRS from Tayca;

alumina and treated with a silicone such as the products TIPAQUE TTO-55(S) from Ishihara or UV TITAN M 262 from Kemira;

triethanolamine such as the product STT-65-S from Titan Kogyo;

stearic acid such as the product TIPAQUE TTO-55(C) from Ishihara; and

sodium hexametaphosphate such as the product MICROTITANIUM DIOXIDE MT 150 W from Tayca.

Other titanium oxide pigments treated with a silicone are preferably TiO₂ treated with octyltrimethylsilane and of which the average size of the elemental particles is from 25 to 40 nm such as that marketed under the trademark T 805 by Degussa Silicas, TiO₂ treated with a polydimethylsiloxane and of which the average size of the elemental particles is 21 nm such as that marketed under the trademark 70250 CARDRE UF TiO₂SI3 by Cardre, anatase/rutile TiO₂ treated with a polydimethylhydrogensiloxane and of which the average size of the elemental particles is 25 nm such as that marketed under the trademark MICROTITANIUM DIOXIDE USP GRADE HYDROPHOBIC by Color Techniques.

The uncoated titanium oxide pigments are, for example, marketed by Tayca under the trademarks MICROTITANIUM DIOXIDE MT 500 B, or MICROTITANIUM DIOXIDE MT 600 B, by Degussa under the trademark P 25, by Wacker under the trademark Transparent Titanium Oxide PW, by Miyoshi Kasei under the trademark UFTR, by Tomen under the trademark ITS and by Tioxide under the trademark TIOVEIL AQ.

The uncoated zinc oxide pigments are, for example:

those marketed under the trademark Z-COTE by Sunsmart;

those marketed under the trademark NANOX by Elementis; and

those marketed under the trademark NANOGARD WCD 2025 by Nanophase Technologies.

The coated zinc oxide pigments are, for example

those marketed under the trademark Z-COTE HP1 by Sunsmart (dimethicone coated ZnO);

those marketed under the trademark Oxide Zinc CS-5 by Toshibi (ZnO coated with polymethylhydrogensiloxane);

those marketed under the trademark NANOGARD ZINC OXIDE FN by Nanophase Technologies (as a 40% dispersion in Finsolv TN, benzoate of C₁₂-C₁₅ alcohols);

those marketed under the trademark DAITOPERSION ZN-30 and DAITOPERSION ZN-50 by Daito (dispersions in oxyethylenated cyclopolymethylsiloxane/polydimethylsiloxane, containing 30% or 50% of nano zinc oxides coated with silica and polymethylhydrogensiloxane);

those marketed under the trademark NFD ULTRAFINE ZnO by Daikin (ZnO coated with perfluoroalkyl phosphate and perfluoroalkylethyl-based copolymer as a dispersion in cyclopentasiloxane);

those marketed under the trademark SPD-Z1 by Shin-Etsu (ZnO coated with silicone-grafted acrylic polymer, dispersed in cyclodimethylsiloxane);

those marketed under the trademark ESCALOL Z100 by ISP (ZnO treated with alumina and dispersed in the ethylhexyl methoxycinnamate/PVP-hexadecene copolymer/methicone mixture);

those marketed under the trademark FUJI ZnO-SMS-10 by Fuji Pigment (ZnO coated with silica and polymethylsilsesquioxane); and

those marketed under the trademark NANOX GEL TN by Elementis (ZnO dispersed to 55% in the benzoate of C₁₂-C₁₅ alcohols with polycondensate of hydroxystearic acid).

The uncoated cerium oxide pigments are marketed, for example, under the trademark COLLOIDAL CERIUM OXIDE by Rhone Poulenc.

The uncoated iron oxide nanopigments are, for example, marketed by Arnaud under the trademarks NANOGARD WCD 2002 (FE 45B), NANOGARD IRON FE 45 BL AQ, NANOGARD FE 45R AQ, NANOGARD WCD 2006 (FE 45R) or by Mitsubishi under the trademark TY-220.

The coated iron oxide pigments are, for example, marketed by Arnaud under the trademarks NANOGARD WCD 2008 (FE 45B FN), NANOGARD WCD 2009 (FE 45B 556), NANOGARD FE 45 BL 345, NANOGARD FE 45 BL or by BASF under the trademark TRANSPARENT IRON OXIDE.

Also exemplary are mixtures of metal oxides, especially of titanium dioxide and cerium dioxide, including the equal-weight mixture of titanium dioxide and cerium dioxide coated with silica, marketed by Ikeda under the trademark SUNVEIL A, and also the mixture of titanium dioxide and zinc oxide coated with alumina, silica and silicone such as the product M 261 marketed by Kemira or coated with alumina, silica and glycerol such as the product M 211 marketed by Kemira.

The additional photoprotective agents are generally present in the compositions according to the invention in amounts ranging from 0.01 to 20% by weight relative to the total weight of the composition, and preferably ranging from 0.1 to 10% by weight relative to the total weight of the composition.

The compositions according to the invention may also contain artificial tanning and/or browning agents for the skin (self-tanning agents) and more particularly dihydroxyacetone (DHA). They are preferably present in amounts ranging from 0.1 to 10% by weight relative to the total weight of the composition.

The aqueous compositions according to the present invention may moreover comprise conventional cosmetic adjuvants in particular selected from fatty substances, organic solvents, ionic or non-ionic, hydrophilic or lipophilic thickeners, demulcents, humectants, opacifiers, stabilizers, emollients, silicones, anti-foaming agents, fragrances, preservatives, anionic, cationic, non-ionic, zwitterionic or amphoteric surfactants, active agents, fillers, polymers, propellants and basifying or acidifying agents, or any other ingredient commonly used in the cosmetics and/or dermatological field.

The fatty substances may be an oil or a wax other than the apolar waxes as defined previously or mixtures thereof. The term “oil” means a compound that is liquid at ambient temperature. The term “wax” means a compound that is solid or substantially solid at ambient temperature, and of which the melting point is generally greater than 35° C.

As oils, exemplary are mineral oils (paraffin); vegetable oils (sweet almond oil, macadamia oil, blackcurrant seed oil, jojoba oil); synthetic oils such as perhydrosqualene, alcohols, fatty amides (such as isopropyl lauroyl sarcosinate marketed under the trademark ELDEW SL-205 by Ajinomoto), fatty acids or esters such as the benzoate of C₁₂-C₁₅ alcohols marketed under the trademark FINSOLV TN or WITCONOL TN by Witco, 2-ethylphenyl benzoate such as the commercial product marketed under the trademark X-TEND 226® by ISP, octyl palmitate, isopropyl lanolate, triglycerides, including those of capric/caprylic acids, dicaprylyl carbonate marketed under the trademark CETIOL CC by Cognis, oxyethylenated or oxypropylenated fatty esters and ethers; silicone oils (cyclomethicone, polydimethylsiloxanes or PDMSs) or fluoro oils, and polyalkylenes.

As waxy compounds, exemplary are carnauba wax, beeswax, hydrogenated castor oil, polyethylene waxes and polymethylene waxes such as that marketed under the trademark CIREBELLE 303 by Sasol.

Among the organic solvents, exemplary are lower polyols and alcohols. The lower polyols may be selected from glycols and glycol ethers such as ethylene glycol, propylene glycol, butylene glycol, dipropylene glycol and diethylene glycol.

As hydrophilic thickeners, exemplary are carboxyvinyl polymers such as carbopols (carbomers) and pemulens (acrylate/C₁₀-C₃₀ alkyl acrylate copolymers); polyacrylamides such as for example the crosslinked copolymers marketed under the trademarks SEPIGEL 305 (CTFA name: polyacrylamide/C₁₃₋₁₄ isoparaffin/Laureth 7) or SIMULGEL 600 (CTFA name: acrylamide/sodium acryloyldimethyltaurate copolymer/isohexadecane/polysorbate 80) by Seppic; polymers and copolymers of 2-acrylamido-2-methylpropanesulfonic acid optionally crosslinked and/or neutralized, such as poly(2-acrylamido-2-methylpropanesulfonic acid) marketed by Hoechst under the trademark HOSTACERIN AMPS (CTFA name: ammonium polyacryloyldimethyl taurate) or SIMULGEL 800 marketed by Seppic (CTFA name: sodium polyacryloyldimethyl taurate/polysorbate 80/sorbitan oleate); copolymers of 2-acrylamido-2-methylpropanesulfonic acid and hydroxyethyl acrylate such as SIMULGEL NS and SEPINOV EMT 10 marketed by Seppic; cellulose derivatives such as hydroxyethyl cellulose; polysaccharides and especially gums such as xanthan gum; and mixtures thereof.

As lipophilic thickeners, exemplary are synthetic polymers such as poly(C₁₀-C₃₀ alkyl acrylates) marketed under the trademark INTELIMER IPA 13-1 and INTELIMER IPA 13-6 by Landec or else modified clays such as hectorite and its derivatives, such as the products marketed under the trademark BENTONE.

Among the active ingredients exemplary are:

vitamins (A, C, E, K, PP, etc.) and their derivatives or precursors, alone or as mixtures;

agents for combating pollution and/or free-radical scavengers;

depigmenting agents and/or propigmenting agents;

anti-glycation agents;

soothing agents;

NO-synthase inhibitors;

agents that stimulate the synthesis of dermal or epidermal macromolecules and/or that prevent their degradation;

agents that stimulate fibroblast proliferation;

agents that stimulate keratinocyte proliferation;

muscle relaxants;

tensioning agents;

mattifying agents;

keratolytic agents;

desquamating agents;

moisturizers;

anti-inflammatory agents;

agents that act on the energy metabolism of cells;

insect repellents;

substance P antagonists or CRGP antagonists;

agents for preventing hair loss and/or for regrowth; and

anti-wrinkle agents.

Of course, one skilled in the art will take care to select the optional additional compound or compounds mentioned above and/or their amounts so that the advantageous properties intrinsically linked to the compositions according to the invention are not, or are not substantially, impaired by the envisaged addition or additions, especially the improvement of the photostability of the dibenzoylmethane derivative.

The compositions according to the invention may be formulated according to the techniques well known to one skilled in the art. They may be, in particular in the form of a simple or complex (O/W, W/O, O/W/O or W/O/W) emulsion such as a cream, a milk or a cream-gel; in the form of an aqueous gel; or in the form of a lotion. They may also be packaged in an aerosol and be in the form of a foam or spray.

Preferably, the compositions according to the invention are in the form of an oil-in-water or water-in-oil emulsion.

The emulsions generally contain at least one emulsifier selected from amphoteric, anionic, cationic or non-ionic emulsifiers, used alone or as a mixture. The emulsifiers are selected in an appropriate manner according to the emulsion to be obtained (W/O or O/W). The emulsions may also contain other types of stabilizers such as for example fillers, gelling polymers or thickeners.

As emulsifying surfactants that can be used for preparing W/O emulsions, mention may be made, for example, of alkyl esters or ethers of sorbitan, of glycerol or of sugars; silicone surfactants, for example dimethicone copolyols, such as the mixture of cyclomethicone and of dimethicone copolyol marketed under the trademark DC 5225 C by Dow Corning, and alkyldimethicone copolyols such as laurylmethicone copolyol marketed under the trademark Dow Corning 5200 Formulation Aid by Dow Corning; cetyl dimethicone copolyol such as the product marketed under the trademark ABIL EM 90R by Goldschmidt and the mixture of cetyl dimethicone copolyol, polyglycerol isostearate (4 mol) and hexyl laurate marketed under the trademark ABIL WE 09 by Goldschmidt. It is possible to also add thereto one or more co-emulsifiers, which advantageously may be selected from the group comprising alkylated polyol esters.

As alkylated polyol esters, especially exemplary are polyethylene glycol esters, for example PEG-30 dipolyhydroxystearate such as the product marketed under the trademark ARLACEL P135 by ICI.

As glycerol and/or sorbitan esters, mention may be made for example of polyglycerol isostearate such as the product marketed under the trademark ISOLAN GI 34 by Goldschmidt; sorbitan isostearate, such as the product marketed under the trademark ARLACEL 987 by ICI; sorbitan isostearate and glycerol such as the product marketed under the trademark ARLACEL 986 by ICI, and mixtures thereof.

For the O/W emulsions, mention may be made, for example as emulsifiers, of non-ionic emulsifiers such as oxyalkylenated (more particularly polyoxyethylenated) fatty acid and glycerol esters; oxyalkylenated fatty acid and sorbitan esters; oxyalkylenated (oxyethylenated and/or oxypropylenated) fatty acid esters such as the PEG-100 stearate/glyceryl stearate mixture marketed, for example by ICI under the trademark ARLACEL 165; oxyalkylenated (oxyethylenated and/or oxypropylenated) fatty alcohol ethers; sugar esters such as sucrose stearate; fatty alcohol and sugar ethers, especially alkylpolyglucosides (APGs) such as decyl glucoside and lauryl glucoside marketed, for example, by Henkel under the respective names PLANTAREN 2000 and PLANTAREN 1200, cetostearyl glucoside, optionally as a mixture with cetostearyl alcohol, marketed, for example under the trademark MONTANOV 68 by Seppic, under the trademark TEGOCARE CG90 by Goldschmidt and under the trademark EMULGADE KE3302 by Henkel, and also arachidyl glucoside, for example, in the form of the mixture of arachidyl and behenyl alcohols and arachidyl glucoside marketed under the trademark MONTANOV 202 by Seppic. According to one particular embodiment of the invention, the mixture of alkyl polyglucoside as defined above with the corresponding fatty alcohol may be in the form of a self-emulsifying composition, as described, for example, in WO-A-92/06778.

Among the other emulsion stabilizers, use will more particularly be made of the polymers of isophthalic acid or sulfoisophthalic acid, and in particular phthalate/sulfoisophthalate/glycol copolymers, for example, the diethylene glycol/phthalate/isophthalate/1,4-cyclohexanedimethanol copolymer (INCI name: Polyester-5) marketed under the trademarks EASTMAN AQ polymer (AQ35S, AQ38S, AQ55S, AQ48 ULTRA) by Eastman Chemical.

When an emulsion is involved, the aqueous phase thereof may comprise a non-ionic vesicular dispersion prepared according to known processes (Bangham, Standish and Watkins, J. Mol. Biol., 13, 238 (1965), FR-2,315,991 and FR-2,416,008).

The compositions according to the invention find application in a large number of treatments, especially cosmetic treatments, whether regime or regimen, for the skin, lips and hair, including the scalp, in particular for protecting and/or caring for the skin, lips and/or hair, and/or for making up the skin and/or the lips.

The present invention also features the use of the compositions according to the invention for the cosmetic treatment of the skin, lips, nails, hair, eyelashes, eyebrows and/or scalp, especially when formulated as care products, sunscreen products and makeup products.

The cosmetic compositions according to the invention may, for example, be used as a makeup product.

The cosmetic compositions according to the invention may, for example, be used as a care product and/or sunscreen product for the face and/or body, of a liquid to semi-liquid consistency, such as milks, more or less rich creams, cream-gels or pastes. They may optionally be packaged in an aerosol and be in the form of a foam or spray.

The compositions according to the invention in the form of vaporizable fluid lotions according to the invention are applied to the skin or hair in the form of fine particles using pressurized devices. The devices according to the invention are well known to one skilled in the art and comprise non-aerosol pumps or “atomizers”, aerosol containers comprising a propellant and also aerosol pumps that use compressed air as a propellant. The latter are described in U.S. Pat. Nos. 4,077,441 and 4,850,517.

The compositions packaged as an aerosol in accordance with the invention generally contain conventional propellants such as, for example, hydrofluoro compounds, dichlorodifluoromethane, difluoroethane, dimethyl ether, isobutane, n-butane, propane or trichlorofluoromethane. They are preferably present in amounts ranging from 15 to 50% by weight relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### SYNTHESIS EXAMPLES

### Example 1

### Preparation of N-(3-{1,3,3,3-tetramethyl-1-[(trimethylsilyl)oxy]disiloxanyl}propyl)benzamide

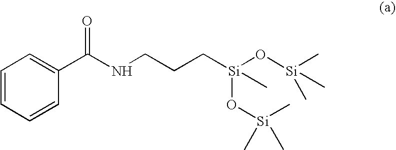

To a solution cooled to 0° C., of benzoic acid (2.32 g, 0.019 mol) in 20 ml of anhydrous dichloromethane and 0.5 ml of DMF, 2 ml of oxalyl chloride (0.0228 mol) were added dropwise. The mixture was stirred for 15 minutes at 0° C. then 3 hours at lab. temperature. The reaction mixture was concentrated under vacuum. The yellow solid obtained was dissolved in 20 ml of dichloromethane and was cooled to 0° C. Added dropwise thereto were 3-aminopropylmethylbis(trimethylsiloxy)silane (5.25 g, 0.019 mol) dissolved in 10 ml of anhydrous dichloromethane, then diisopropylethylamine (9.5 ml, 0.057 mol). After evaporating the solvent under vacuum, the orange oil obtained was taken up in 100 ml of ethyl acetate. After washing the organic phase twice with water, then with a saturated solution of sodium chloride, it was dried over sodium sulfate. After filtration and evaporation of the solvent, the orange oil obtained was chromatographed on silica (eluent: heptane/EtOAc:95/5) to give 5.1 g (yield: 70%) of a pale yellow oil of the compound of Example 1.

### Example 2

### Preparation of 3-phenyl-N-(3-{1,3,3,3-tetramethyl-1-[(trimethylsilyl)oxy]disiloxanyl}propyl)propanamide

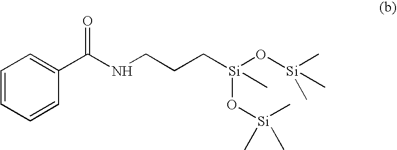

To a solution cooled to 0° C., of 3-phenylpropionic acid (11 g, 0.0732 mol) in 40 ml of anhydrous acetonitrile and 1 ml of DMF, 7.7 ml of oxalyl chloride (0.0878 mol) were added dropwise. The mixture was stirred for 15 minutes at 0° C. then 3 hours at lab. temperature. The reaction mixture was concentrated under vacuum. The orange oil obtained was dissolved in 40 ml of acetonitrile and was cooled to 0° C. Added dropwise thereto were 3-aminopropylmethylbis(trimethylsiloxy)silane (20.48 g, 0.0732 mol) dissolved in 40 ml of anhydrous acetonitrile, then diisopropylethylamine (37 ml, 0.219 mol). After evaporating the solvent under vacuum, the orange oil obtained was taken up in 300 ml of ethyl acetate. After washing the organic phase twice with water, then with a saturated solution of sodium chloride, it was dried over sodium sulfate. After filtration and evaporation of the solvent, the orange oil obtained was chromatographed on silica (eluent: heptane/EtOAc:95/5) to give 23 g (yield: 76%) of a yellow oil of the compound of Example 2.

### Formulation Examples 3 and 4

The following compositions were produced, then for each of them the photostability of 4-tert-butyl-4′-methoxydibenzoylmethane was evaluated.

| | Example 3 | |
|---|---|---|
| Compositions | (comparative) | Example 4 |
| Polydimethylsiloxane | 0.5 | 0.5 |
| Preservatives | 1 | 1 |
| Stearic acid | 1.5 | 1.5 |
| Glycerylmonostearate/PEG stearate | 1 | 1 |
| (100 EO) mixture (ARLACEL P165 ® | | |
| - UNIQUEMA) | | |
| Sequestering agent | 0.1 | 0.1 |
| C₁₂-C₁₅ alkyl benzoate (FINSOLV | 15 | — |
| TN ® from Witco) | | |
| Compound of Example 1 (Compound | | 15 |
| a) | | |
| 4-tert-butyl- | 2 | 2 |
| 4′methoxydibenzoylmethane | | |
| (PARSOL 1789 ® DSM Nutritional | | |
| Products) | | |
| Glycerol | 5 | 5 |
| Xanthan gum | 0.2 | 0.2 |
| Potassium Cetyl Phosphate | 1 | 1 |
| (AMPHISOL K ® - DSM Nutritional | | |
| Products) | | |
| Isohexadecane | 1 | 1 |
| Acrylic acid/stearyl | 0.2 | 0.2 |
| methacrylate copolymer | | |
| (PEMULEN TR 1 ® - Noveon) | | |
| Cetyl alcohol | 0.5 | 0.5 |
| Triethanolamine | 0.65 | 0.65 |
| Mixture of cetylstearyl glucoside and | 2 | 2 |
| cetyl and stearyl alcohols | | |
| (MONTANOV 68 ® - Seppic) | | |
| Deionized water | qs for 100 | qs for 100 |

Measurement Method:

For each formula, 4 test samples and 4 control samples were prepared. 2 mg/cm² of formula were deposited, using a spatula, on polymethyl methacrylate sheets.

The test sheets were exposed for 46 min to the Heraus Suntest provided by a xenon lamp having a UV-A flux of 7.9×10⁻³ W/cm² and a UV-B flux of 3.8×10⁻⁴ W/cm². The control sheets were stored for the same time and at the same temperature (38-40° C.) in darkness. At the end of this time, the screening agents were removed by immersing each sheet in 50 g of ethanol and by subjecting them to ultrasound for 15 min in order to ensure a good removal. The solutions obtained were analyzed by UV spectrophotometry. For each formula tested, the residual 4-tert-butyl-4′-methoxydibenzoylmethane level after exposure was given in the ratio of its optical density (OD) in the exposed sample to its unexposed optical density (OD). This took place at the absorption maximum corresponding to butylmethoxydibenzoylmethane: λₘₐₓ=358 nm.

The results obtained are summarized in the table below:

| | Residual % of dibenzoylmethane after |
|---|---|
| Compositions | exposure to UV-A for 1 hour |
| Formula 3 | 27.7 ± 6 |
| Formula 4 (invention) | 44.7 ± 6 |

An improvement in the photostability of the dibenzoylmethane was observed in the presence of siloxane-containing arylalkyl amide derivatives.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable photostabilized cosmetic/dermatological composition comprising at least one dibenzoylmethane compound UV-A screening agent and at least one siloxane-containing arylalkyl benzoate amide compound of formula (I) below: 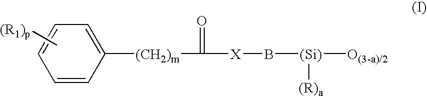
in which
R₁ is a linear or branched C₁-C₄ alkyl radical;
p is 0-2;
m is 0-4;
X is —N(R₂)—
R₂ is H, a linear or branched C₁-C₂₀ alkyl radical, the acetyl group or the group —CH₂CH₂NH(C═O)Ph;
the radicals R, which may be identical or different, are each a linear or branched C₁-C₂₀ alkyl radical, a C₆-C₁₂ aryl radical, 3,3,3-trifluoropropyl, trimethylsilyloxy or a linear or branched C₁-C₁₀ alkoxy radical;
a=1-2;
B is a divalent radical selected from among those having one of the formulae (II), (III) or (IV) below: 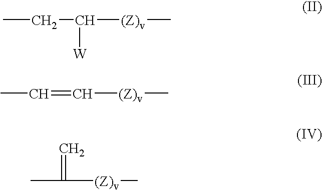
in which
Z is a linear or branched, saturated or unsaturated C₁-C₆ alkylene radical, optionally substituted by a hydroxyl radical or a linear or branched, saturated or unsaturated C₁-C₈ alkyl radical;
W is a hydrogen atom, a hydroxyl radical or a linear or branched, saturated or unsaturated C₁-C₈ alkyl radical; and
v is 0 or 1, formulated into a topically applicable, cosmetically/dermatologically acceptable support therefor.

2. The cosmetic/dermatological composition as defined by claim 1, wherein the compound of the formula (I) comprises structural units of formula (R)_{b}—(Si)(O)_{(4-b)/2} in which:
R has the same definition; and
b=2-3.

3. The cosmetic/dermatological composition as defined by claim 2, wherein formula (I) the —(Si)(R)ₐ(O)_{(3-a)/s} groups have the formulae (V) or (VI) below: 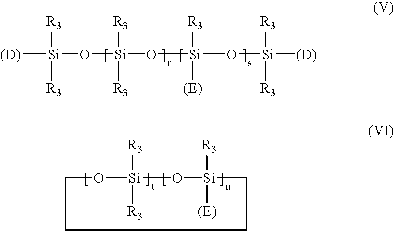
in which:
(E) is a bond from the silicone-based chain and the group B in the formula (I);
the radicals R₃, which may be identical or different, are each selected from among the linear or branched C₁-C₂₀ alkyl radicals, phenyl, 3,3,3-trifluoropropyl and trimethylsilyloxy radicals, at least 80% by number of the R₃ radicals being methyl;
the radicals (D) which may be identical or different, are each selected from among the R₃ radicals and the (E) radical;
r is an integer ranging from 0 to 200 inclusively, and s is an integer ranging from 0 to 50 inclusively, and when s=0, at least one of the two symbols (D) is B,
u is an integer ranging from 1 to 10 inclusively, and t is an integer ranging from 0 to 10 inclusively, with the proviso that t+u is greater than or equal to 3, with the condition that when q is 1, then u is equal to 1, s is equal to 1 and the radical (D) cannot denote B or s is equal to 0 and only one of the radicals (D) is B.

4. The cosmetic/dermatological composition as defined by claim 3, wherein the linear or cyclic diorganosiloxane of formula (V) or (VI) is a random oligomer or polymer having at least one of the following characteristics:
R₃ is methyl; and
(D) is methyl.

5. The cosmetic/dermatological composition as defined by claim 1, wherein the compound of formula (I) is selected from among the following compounds of formulae (a) to (e):

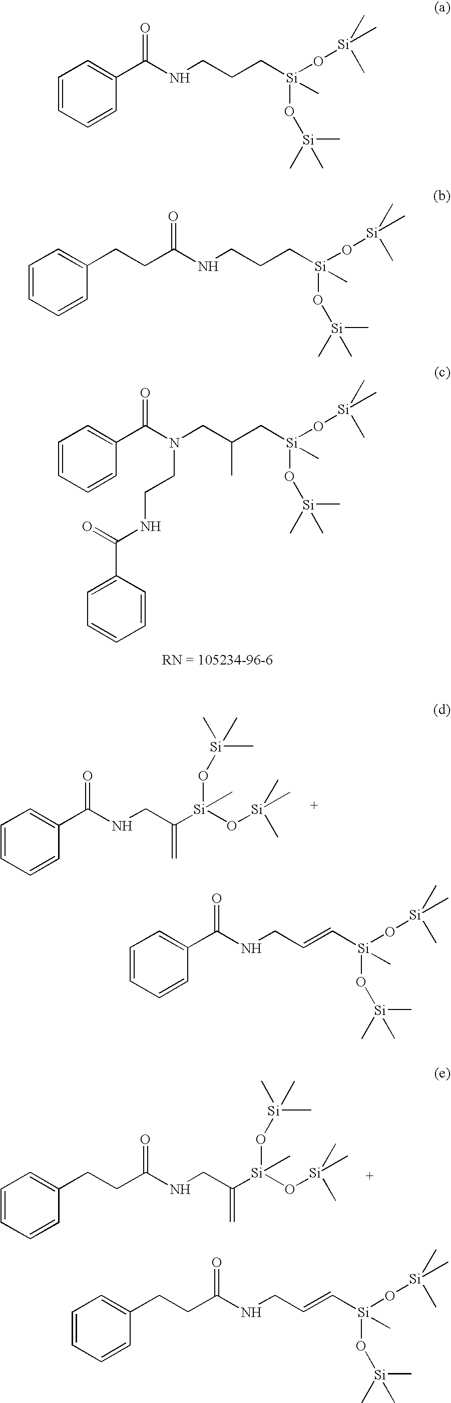

6. The cosmetic/dermatological composition as defined by claim 1, wherein the siloxane-containing arylalkyl benzoate amide compound or compounds of formula (I) are present therein in amounts of 1 to 30% by weight relative to the total weight of the composition.

7. The cosmetic/dermatological composition as defined by claim 1, said at least one dibenzoylmethane compound being selected from among:
2-methyldibenzoylmethane;
4-methyldibenzoylmethane;
4-isopropyldibenzoylmethane;
4-tert-butyldibenzoylmethane;
2,4-dimethyldibenzoylmethane;
2,5-dimethyldibenzoylmethane;
4,4′-diisopropyldibenzoylmethane;
4,4′-dimethoxydibenzoylmethane;
4-tert-butyl-4′-methoxydibenzoylmethane;
2-methyl-5-isopropyl-4′-methoxydibenzoylmethane;
2-methyl-5-tert-butyl-4′-methoxydibenzoylmethane;
2,4-dimethyl-4′-methoxydibenzoylmethane; and
2,6-dimethyl-4-tert-butyl-4′-methoxydibenzoylmethane.

8. The cosmetic/dermatological composition as defined by claim 7, wherein the at least one dibenzoylmethane compound comprises 4-isopropyldibenzoylmethane having the following formula:

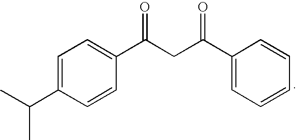

9. The cosmetic/dermatological composition as defined by claim 7, wherein the at least one dibenzoylmethane compound comprises 4-(tert-butyl)-4′-methoxydibenzoylmethane or Butyl Methoxy Dibenzoylmethane having the following formula:

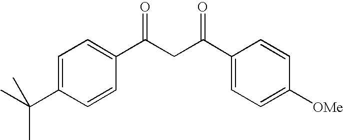

10. The cosmetic/dermatological composition as defined by claim 1, wherein the dibenzoylmethane compound or compounds are present in amounts which range from 0.01 to 10% by weight relative to the total weight of the composition.

11. The cosmetic/dermatological composition as defined by claim 1, further comprising other organic or inorganic photoprotective agents that are active in the UV-A and/or UV-B range and that are water-soluble or liposoluble or insoluble in the conventional cosmetic solvents.

12. The cosmetic/dermatological composition as defined by claim 11, comprising additional organic photoprotective agents selected from among anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis(benzoazolyl) derivatives; p-aminobenzoic acid (PABA) derivatives; methylene bis(hydroxyphenylbenzotriazole) derivatives; benzoxazole derivatives; screening polymers and screening silicones; α-alkylstyrene-derived dimers; 4,4-diarylbutadienes and mixtures thereof.

13. The cosmetic/dermatological composition as defined by claim 12, comprising additional organic UV screening agent(s) selected from among the following compounds:
Ethylhexyl Methoxycinnamate;
Homosalate;
Ethylhexyl Salicylate;
Octocrylene;
Phenylbenzimidazole Sulfonic Acid;
Benzophenone-3;
Benzophenone-4;
Benzophenone-5;
n-Hexyl 2-(4-diethylamino-2-hydroxybenzoyl)benzoate;
4-Methylbenzylidene camphor;
Terephthalylidene Dicamphor Sulfonic Acid;
Disodium Phenyl Dibenzimidazole Tetrasulfonate;
Methylene bis-Benzotriazolyl Tetramethylbutylphenol;
Ethylhexyl triazone,
Bis-Ethylhexyloxyphenol Methoxyphenyl Triazine;
Diethylhexyl Butamido Triazone;
2,4,6-Tris(dineopentyl 4′-aminobenzalmalonate)-s-triazine;
2,4,6-Tris(diisobutyl 4′-aminobenzalmalonate)-s-triazine;
2,4,6-Tris(biphenyl-4-yl-1,3,5-triazine);
2,4,6-Tris(terphenyl)-1,3,5-triazine;
Drometrizole Trisiloxane;
Polysilicone-15;
Dineopentyl 4′-methoxybenzalmalonate
1,1-Dicarboxy-(2,2′-dimethylpropyl)-4,4-diphenylbutadiene;
2,4-Bis[5-1 (dimethylpropyl)benzoxazol-2-yl-(4-phenyl)-imino]-6-(2-ethylhexyl)-imino-1,3,5-triazine;
and mixtures thereof.

14. The cosmetic/dermatological composition as defined by claim 11, comprising additional inorganic photoprotective agents selected from among treated or untreated metal oxide pigments.

15. The cosmetic/dermatological composition as defined by claim 14, said pigments comprising treated or untreated titanium, zinc, iron, zirconium or cerium oxides and mixtures thereof.

16. The cosmetic/dermatological composition as defined by claim 1, further comprising at least one artificial tanning and/or browning agent for the skin.

17. The cosmetic/dermatological composition as defined by claim 1, further comprising at least one adjuvant selected from among fatty substances, organic solvents, ionic or non-ionic, hydrophilic or lipophilic thickeners, demulcents, humectants, opacifiers, stabilizers, emollients, silicones, anti-foaming agents, fragrances, preservatives, anionic, cationic, non-ionic, zwitterionic or amphoteric surfactants, active agents, fillers, polymers, propellants and basifying or acidifying agents.

18. The cosmetic/dermatological composition as defined by claim 1, formulated as an oil-in-water or water-in-oil emulsion.

19. The cosmetic/dermatological composition as defined by claim 1, formulated as a product for the cosmetic treatment of the skin, lips, nails, hair, eyelashes, eyebrows and/or scalp.

20. The cosmetic/dermatological composition as defined by claim 1, formulated as a care product for the skin, lips, nails, hair and/or scalp.

21. The cosmetic/dermatological composition as defined by claim 1, formulated as a makeup product.

22. A process for improving the chemical stability with respect to UV radiation of at least one dibenzoylmethane compound UV-A screening agent, comprising formulated therewith an effective amount of at least one siloxane-containing arylalkyl benzoate amide compound.

23. A regime or regimen for photoprotecting a keratinous substrate against the damaging effects of UV-A radiation, comprising topically applying thereon a thus effective amount of the cosmetic/dermatological composition as defined by claim 1.

24. A regime or regimen for photoprotecting the skin, hair, lips, scalp, nails, eyelashes and/or eyebrows against the damaging effects of UV-A radiation, comprising topically applying thereon a thus effective amount of the cosmetic/dermatological composition as defined by claim 1.
